(19) United States
(12) Patent Application Publication
(10) Pub. No.: US 2025/0069725 A1
(43) Pub. Date: Feb. 27, 2025

(54) SYSTEM AND METHOD FOR TRAINING, TAGGING, RECOMMENDING, AND GENERATING DIGITAL CONTENT BASED ON BIOMETRIC DATA

(71) Applicant: StoryUp, Inc., Columbia, MO (US)

(72) Inventor: Sarah E. Hill, Columbia, MO (US)

(21) Appl. No.: 18/811,687

(22) Filed: Aug. 21, 2024

Related U.S. Application Data
(60) Provisional application No. 63/520,989, filed on Aug. 22, 2023.

(51) Int. Cl. G16H 20/70

(52) U.S. Cl. CPC G16H 20/70

(57) ABSTRACT

The disclosure relates to systems and methods for training, tagging, recommending and/or generating digital content, such as but not limited to virtual reality (“VR”), augmented reality (“AR”), mixed reality (“MR”) content, audio content, spatial audio content, video content (e.g., 2-dimensional (“2D”) and/or flat video content), audiovisual content, and/or any combination thereof, based on user biometrics. The systems and methods are adapted to process the biometric data of a user to evaluate a biometric response of the user to the digital content. The systems and methods are further adapted to modify or generate, based on the biometric response of the user, digital content to improve one or more metrics indicative of the physiological health, psychiatric health, general fitness, and/or wellness of the user.

### CROSS-REFERENCE TO RELATED APPLICATIONS

This application claims priority to U.S. Provisional Patent Application Ser. No. 63/520,989, filed on Aug. 22, 2023, to Sarah E. Hill, entitled “System and Method for Training, Tagging, and Recommending Content By Bio-Data States,” the entire disclosure of which is incorporated herein by reference.

In addition, the entire disclosures of U.S. patent application Ser. No. 16/286,822, now issued as U.S. Pat. No. 11,101,031, filed on Feb. 27, 2019, to Sarah E. Hill et al. entitled “System and method for modifying biometric activity using virtual reality therapy,” and U.S. patent application Ser. No. 15/862,478, now issued as U.S. Pat. No. 10,347,376, filed on Jan. 4, 2018, to Sarah E. Hill et al. entitled “System and method for modifying biometric activity using virtual reality therapy,” are incorporated herein by reference.

### BACKGROUND OF THE INVENTION

Stress is the 21st century epidemic and a $300 billion profit killer that leads to burnout, reduced productivity, employee turnover, errors, poor decision-making, and even death by suicide. With the rise of the opioid epidemic, people are more mindful of addiction and not as quick to take pharmaceutical drugs to address common issues. Video, audio, and other 2-dimensional (2D) and/or 3-dimensional (3D) digital content can mimic the impact of some pain and anxiety medications without the side effects or addiction worries of pharmaceuticals. Content creators currently do not understand how the content they are creating impacts viewers' physiology because there is minimal objective data where viewers are connected to health monitoring equipment while watching content.

Accordingly, a need exists for new approaches in treating psychological and psychiatric conditions, such as a user's anxiety, depression, stress, post-traumatic stress disorder (PTSD), phobias, and pain control.

### SUMMARY

The present invention is directed to systems and methods for training, tagging, recommending and/or generating digital content, such as but not limited to virtual reality (“VR”), augmented reality (“AR”), mixed reality (“MR”) content, audio content, spatial audio content, video content (e.g., 2-dimensional (“2D”) and/or flat video content), audiovisual content, and/or any combination thereof, based on user bio-data. With the disclosed techniques, the systems and methods according to the present invention are adapted to process the biometric data of a user to analyze the user's response to digital content. Moreover, with the disclosed techniques, the systems methods according to the present invention are adapted to process and/or generate digital content to improve one or more metrics indicative of a user's physiological health, psychiatric health, and/or general fitness and/or wellness.

According to one embodiment, a method for evaluating a biometric response of a user to digital content is presented. The method includes collecting, via a biometric tracker, a first biometric data of the user, wherein the biometric tracker includes at least one sensing device; processing the first biometric data to establish a baseline dataset for the user; extracting at least one feature parameter from the digital content; collecting, via the biometric tracker, a second biometric data of the user while the user is exposed to the digital content; generating, via an iteratively trained training model, a model output based on the second biometric data and the at least one feature parameter; determining an impact score for the digital content based on the baseline dataset and the model output; and presenting, via a display device, the impact score to the user.

According to another embodiment, a system for collecting evaluating biometric data from a user while the user is exposed to digital content is provided. The system includes a biometric tracker including a sensor, wherein the biometric tracker is designed to collect biometric data of the user; a device adapted to present the digital content to the user; and a controller including a processor. The processor is configured to process a first biometric data of the user collected by the biometric tracker to establish a baseline dataset for the user; extract at least one feature parameter from the digital content; collect a second biometric data of the user collected by the biometric tracker while the user is exposed to the digital content; process the second biometric data and the at least one feature parameter using an iteratively trained training module; determine an impact score for the digital content based on the baseline dataset and an output of the iteratively trained training module; and present, via the device, the impact score to the user.

### BRIEF DESCRIPTION OF THE SEVERAL VIEWS OF THE DRAWINGS

In the accompanying drawings, which form a part of the specification and are to be read in conjunction therewith in which like reference numerals are used to indicate like or similar parts in the various views:

FIG. 1 is a schematic representation of a system for utilizing digital content as therapeutic treatment for psychological conditions, psychiatric conditions, and/or other general fitness and/or wellness purposes in accordance with one embodiment of the present invention;

FIG. 2 is a schematic representation of a system architecture for utilizing digital content as therapeutic treatment for psychological conditions, psychiatric conditions, and/or other general fitness and/or wellness purposes in accordance with one embodiment of the present invention;

FIG. 3 is a schematic representation and flowchart of a method for utilizing digital content as therapeutic treatment for psychological conditions, psychiatric conditions, and/or other general fitness and/or wellness purposes in accordance with one embodiment of the present invention;

FIG. 4 is a schematic representation and flowchart of a for utilizing digital content as therapeutic treatment for psychological conditions, psychiatric conditions, and/or other general fitness and/or wellness purposes in accordance with one embodiment of the present invention;

FIG. 5A is a graphical representation of example biometric data mapped to digital content in order to train an iteratively trained training module in accordance with one embodiment of the present invention;

FIG. 5B is an illustration of a digital content output in accordance with one embodiment of the present invention;

FIG. 5C is an illustration of a digital content output in accordance with one embodiment of the present invention;

FIG. 6 is an illustration of a digital content output in accordance with one embodiment of the present invention; and

FIG. 7 is a schematic representation of an alternative system architecture that can be implemented in conjunction with the systems and methods of FIGS. 2-6, in accordance with one embodiment of the present invention.

### DETAILED DESCRIPTION OF THE INVENTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures. It will be appreciated that any dimensions included in the drawing figures are simply provided as examples and dimensions other than those provided therein are also within the scope of the invention.

The following detailed description of the invention references specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The present invention is defined by the appended claims and the description is, therefore, not to be taken in a limiting sense and shall not limit the scope of equivalents to which such claims are entitled.

The present invention is directed generally to a system and method for tagging, training, recommending, and/or generating digital content, such as but not limited to, virtual reality (“VR”), augmented reality (“AR”), mixed reality (“MR”) content, audio content, spatial audio content, video content, audiovisual content, or any combination thereof. The systems and methods described herein can be used for wellness, meditation, and in the therapeutic treatment of psychological, psychiatric, or other medical conditions in patients. The present invention is also directed generally to systems and methods for providing specific digital content, such as but not limited to VR and/or AR content, to users as a therapeutic prescription to effect positive changes in the user's biometrics associated with emotional, psychological, and psychiatric states. The systems and methods of the present invention can be configured to provide digital content experiences (e.g., VR, AR, MR, and/or other digital content experiences), including environments and stories, in which the digital content is specifically tailored to shift specific biometrics of a particular user to mitigate patterns associated with anxiety, depression, stress, PTSD, phobias, pain control, or other psychological or psychiatric conditions and promote patterns associated with relaxation, positive affect, and pro-social emotional states. The systems described herein can further be used to provide an immersive digital content experience for the adjunctive treatment of patients to reduce acute anxiety symptoms, including those diagnosed of Generalized Anxiety Disorder (GAD). In some non-limiting embodiments, the systems and methods described herein can be used in applications with virtual reality equipment (e.g., goggles), as well as with standard 2D video and/or applications without the use of goggles, third-party equipment, or devices.

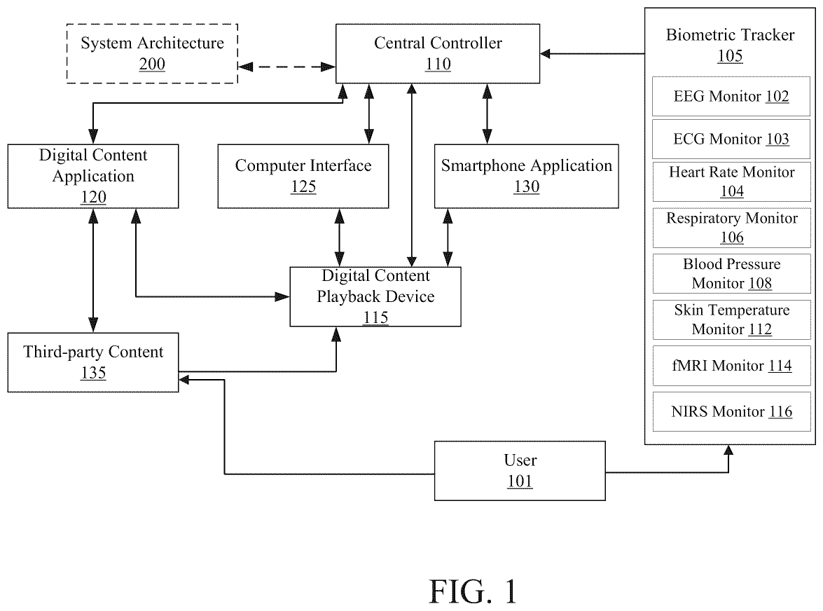

FIG. 1 shows a schematic representation of a system 100 for utilizing digital content as therapeutic treatment for psychological conditions, psychiatric conditions, and/or other general fitness and/or wellness purposes in accordance with some embodiments of the present invention according to one embodiment. The system 100 is configured to present digital content to a user and monitor the user's biometrics resulting from the provided digital content.

As described herein, the term digital content can refer to any one or more of VR content, AR content, MR content, audio content, spatial content, video content, 2D video content, flat or non-spatial video content, and/or audiovisual content. In that regard, the system 100 is configured to provide VR content, AR content, MR content, audio content, spatial content, video content, and/or audiovisual content to a user and monitor the user's biometrics resulting from the provided VR content, AR content, MR content, audio content, spatial content, video content, and/or audiovisual content.

Although the foregoing description of the present invention may refer individually to the use of VR components and concepts in connection with system 100 and methods 300 and 400 disclosed herein; persons skilled in the art will recognize that AR components, MR components, audio components, spatial components, video components, 2-D video components, flat video components, and/or audiovisual components and concepts can just as suitably be used in place of or in combination with VR components and concepts. In accordance with the principles of this disclosure, video content can include both 2D and 3D video content. Moreover, as used herein, a user 101 can refer to a patient, particularly when the system 100 is used as a medical solution for one or more clinical conditions or a treatment for a diagnosis, or more generally to anyone using the system 100 for general health and wellness purposes.

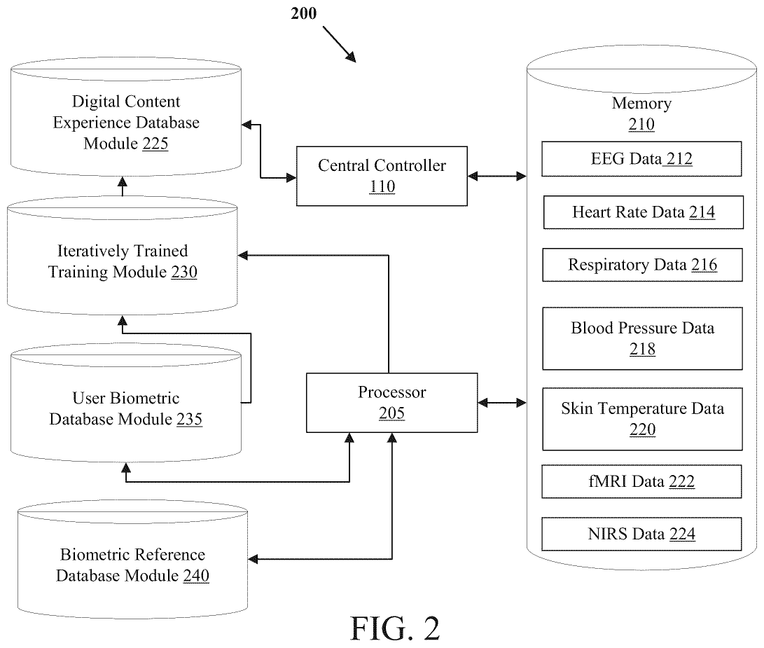

The system 100 can include one or more biometric trackers 105, a central controller 110, and a digital content playback device 115 according to certain embodiments of the present invention. The central controller 110, which may be implemented as any suitable computing device (e.g., desktop computer, laptop, server, smartphone, tablet, smart television, etc.) and/or cloud-based computing device, can include one or more electronic processors adapted to execute programmable instructions to perform processes described with respect to the system architecture 200 of FIG. 2. The one or more biometric trackers 105 receive biometric data associated with a user 101 and pass said biometric data to a processor of the central controller 110, or other aspect of the system 100. The central controller 110 can operatively communicate with a digital content playback device 115, a digital content application 120, a computer interface 125, and/or a smartphone application 130. In some embodiments, the central controller 110 can receive information and data from one or more of the digital content application 120, computer interface 125, and smartphone application 130. In some embodiments, the central controller 110 can generate digital content (e.g., VR content) and transmit the content to be displayed on one more of the digital content application 120, the computer interface 125, and the smartphone application 130. In some embodiments, the digital content playback device 115 can be designed to receive and display third-party content 135 and/or digital content received from the central controller 110. In some embodiments, the digital content can be generated and output to the digital content playback device 115 via one or more of the digital content application 120, computer interface 125, or smartphone application 130. In some embodiments, the digital content application 120 and/or the smartphone application 130 can be executed by one or more processors of the central controller 110.

Hereinafter, the one or more biometric trackers 105 may be referred to as “the biometric tracker 105.” The biometric tracker 105 can be adapted to monitor one or more biometric data of the user 101. Biometric data of a user can include, without limitation, heart rate, heart rate variability (HRV), blood volume controlled by the heart's pumping actions, blood oxygen saturation, blood pressure, the rise and fall of a user's chest, and/or any other biometric data associated with a user. In some examples, the biometric tracker 105 can include one or more of an electroencephalogram (EEG) monitor 102, an electrocardiogram (ECG) monitor 103, a heart rate monitor 104, a respiratory monitor 106, a blood pressure monitor 108, a skin temperature monitor 112, a functional magnetic resonance imaging (fMRI) monitor 114, and/or a near-infrared spectroscopy (NIRS) monitor 116. In some embodiments, the biometric tracker 105 can be a single device capable of capturing data associated with multiple biometric parameters of the user. In some embodiments, the biometric tracker 105 cancan be provided in the form of an integrated device with multiple sensing devices, wherein each sensing device can be designed to collect biometric data associated with one or more biometric parameters. In some embodiments, the biometric tracker 105 can be provided in the form of a personal consumer electronic or wearable monitoring device like a fitness tracker, smartwatch, smart ring, etc. In some embodiments, the biometric tracker 105 can be provided in the form of a pair of headphones that includes one or more integrated biometric sensors, a pair of earbuds that includes one or more integrated biometric sensors, and/or some other wearable device that includes biometric sensors.

In some embodiments, the system 100 utilizes the biometric tracker 105 to monitor, record, and collect certain types of biometric data of a user 101 before, during, and/or after the user engages with digital content. In some embodiments, an iteratively trained training module 230 (which is further discussed with reference to FIG. 2) can be trained based on the percentage shifts in the biometric data before, during, and/or after the user engages with selected digital content (which can indicate, for example, changes in the user's 101 brainwaves in response to viewing digital content). The iteratively trained training module 230 can be adapted to score the selected digital content based on a percentage shift determined in order to generate and provide tailored digital content. In some embodiments, the system 100 can provide personalized recommendations to the user 101 related to digital content that should be selected in order to adjust one or more of the user's 101 sensed biometric parameters. In some embodiments, the system 100 can automatically select the digital content, such as but not limited to a customized VR experience, based on biometric data of the user 101 and output(s) of the iteratively trained training module. In some embodiments, the system 100 can alter the story arc and/or narrative of digital content being played back to a user 101 based on a percentage shift in the biometric data of the user 101.

When used throughout the present disclosure, one skilled in the art will understand that processes for “iteratively training the machine learning training model” can include machine learning processes and other similar advanced artificial intelligence processes. One skilled in the art will understand that terms such as “biometrics” and “biometric data” can include any features, parameters, components, elements, structures, or other particulars pertaining to any biological and/or behavioral characteristics of a user 101 or patient, including but not limited to, electroencephalogram (EEG) readings, heart rate, blood pressure, respiratory patterns, skin temperature, and skin conductance.

According to one embodiment, the biometric tracker 105 includes an EEG monitor 102 to monitor the electrical activity of a user's brain. In some embodiments, the EEG monitor 102, can be provided in the form of a brain computer interface (BCI) and can be configured as a standalone sensing device or as an aspect of a fitness tracker or similar wearable device (e.g., a smartwatch, a fitness tracking ring, etc.). In some embodiments, the EEG monitor 102 can be provided in the form of a camera sensor adapted to detect photoplethysmography and/or tiny pulsations in the skin of the user 101. In some embodiments, the EEG monitor 102 can be provided in the form of an EEG headband, EEG electrocap, EEG earbuds, or similar EEG device, that is placed on or around the head of the user 101 of system 100. The EEG headband, cap, or earbuds can include a plurality of electrodes designed to measure brainwave activity. The EEG headband, cap, or earbuds can be attached via a wire ribbon to an amplifier that monitors and records the electrical activity of the user's brain. Depending on the particular embodiment, the EEG monitor 102 can be configured to monitor one or more brainwave frequencies, including but not limited to alpha, beta, delta, gamma, and theta, in one or more selected regions of the brain of a user 101. The monitoring technique only records electrical signals so there is no risk to the user aside from possible mild discomfort from wearing the EEG headband or cap.

In particular, the EEG monitor 102 records electrical activity produced by different amounts and combinations of neurons firing in a user's brain. Resulting EEG data can be represented by channels (e.g., digital signals recorded by the amplifiers) for each electrode. The signals can comprise different frequencies that are derived via a time-frequency analysis. For example, delta waves can range from 1-3 cycles per second (Hz), theta waves can range from 4-7 Hz, alpha waves can range from 8-12 Hz, and beta waves can range from 13 Hz and higher.

In accordance with the principles of this disclosure, EEG data can be captured by any file format system known to those skilled in the art, such as, but not limited to, BIOSEMI™ ActiveTwo/ActiView (.bdf), BRAIN PRODUCTS™/BRAIN VISION™ (.vhdr, .vmrk, .eeg), European data format (.edf), General data format (.gdf), NEUROSCAN® CNT (.cnt), EGI simple binary (.egi), EGI MFF (.mff), EEGLAB™ files (.set, .fdt), NICOLET® (.data), eXimia EEG data (.nxe), PERSYST® EEG data (.lay, .dat), NIHON KOHDEN® EEG data (.eeg, .21e, .pnt, .log), and XDF data (.xdf, .xdfz). It will be appreciated by those skilled in the art that other file formats are contemplated within the scope of the present disclosure.

According to one embodiment, the biometric tracker 105 can further comprise a heart rate monitor 104 configured to monitor the heart rate variability of the user 101. The heart rate monitor 104 can be provided in the form of an aspect of a fitness tracker or similar wearable device (e.g., a smartwatch, a chest monitor) strapped around a portion of a user's 101 anatomy, and can record heart signals optically, electrically, or otherwise. An optical heart rate monitor can utilize photoplethysmography (PCG) sensors that measure the blood volume controlled by the heart's pumping actions. Such monitors shine from a light-emitting diode (LED) through the skin of a user 101 and detect how the light scatters off underlying blood vessels. In some embodiments, the PCG sensors can also measure blood oxygen saturation (SpO₂) levels. In an alternative embodiment, the heart rate monitor 104 can be an electrical monitor with electrocardiogramaensors that measure the bio-potential generated by electrical signals that control the expansion and contraction of heart chambers. In this embodiment, the heart rate monitor 104 can include a transmitter strapped to the user 101 in the form of a chest strap, and a receiver, wherein a detected heartbeat is transmitted as a radio signal and displayed by the receiver as the current heart rate.

Heart rate variability can be determined from the heart rate data obtained from the heart rate monitor 104 via time-domain methods in bpm (e.g., the extent to which the heart rate changes within a set amount of time) or frequency-domain methods in milliseconds (e.g., the extent to which the heart beating is spread over different frequencies). In accordance with the principles of this disclosure, heart rate data can be captured and processed in one or more file formats or file systems.

According to one embodiment, the biometric tracker 105 can further comprise a respiratory monitor 106 configured to monitor the breathing patterns of the user 101. In some examples, the respiratory monitor 106 can be provided in the form of an aspect of a fitness tracker or similar wearable device (e.g., a smartwatch) strapped around a user's wrist or chest. Alternatively, the respiratory monitor 106 can be a separate device (e.g., a pneumography) having one or more pressure sensors that are strapped around a user's chest or abdomen. The respiratory monitor 106 can include a sensor to detect chest or abdominal expansion and contraction and generate a respiration waveform based on the changing force applied to the sensor while the user 101 is breathing. In some embodiments, the respiratory monitor 106 can be implemented as a camera sensor adapted to detect the rise and fall of a user's chest. In some embodiments, the respiratory monitor 106 can be implemented as an audio sensor (e.g., a microphone) adapted to detect the sound of a user's 101 breathing. In other embodiments, respiratory data can be obtained from one or more pressure sensors incorporated into a facemask (or similar) to measure the force produced by the respiratory tract while the user 101 is breathing. In other embodiments, acoustic, humidity, oximetry, acceleration, resistive, ECG, and/or PPG sensors can be incorporated into the biometric tracker 105 to determine respiratory metrics. In some embodiments, the respiratory monitor 106 measures a user's breathing rate in Hz, and features related to time, frequency, and cepstral can be extracted from the signals obtained. In accordance with the principles of this disclosure, respiratory data can be captured and processed in one or more file formats or file systems.

According to one embodiment, the biometric tracker 105 can further comprise a blood pressure monitor 108. The blood pressure monitor 108 can be provided in the form of an aspect of a fitness tracker or other wearable device (e.g., a smartwatch, ring, headband, etc.) strapped around a user's wrist, chest, finger, head, upper arm, or some other body part. Alternatively, the blood pressure monitor 108 can be provided in the form of a separate device (e.g., manual sphygmomanometer, digital pressure sensors, etc.) strapped around the upper arm, wrist, or finger of a user 108. In some embodiments, the blood pressure monitor 108 measures mean blood pressure, pulse rate, and systolic and diastolic pressures via oscillometric detection using electronic pressure sensors. In some embodiments, the blood pressure monitor 108 uses auscultatory detection with a stethoscope and a sphygmomanometer. Blood pressure data can be measured in units of mmHg (or any other suitable units) for the cuff pressure and oscillation amplitude over time. In accordance with the principles of this disclosure, blood pressure data can be captured and processed in one or more file formats or file systems.

According to one embodiment, the biometric tracker 105 can further comprise a skin conductance and temperature monitor 112 configured to monitor variations in the external temperature of the user 101. The skin temperature monitor 112 can be provided in the form of an aspect of a fitness tracker or other wearable device (e.g., a smartwatch) strapped around a user's 101 wrist. In some embodiments, the skin temperature monitor 112 can be provided in the form of a separate device such as an electronic thermometer or infrared thermometer strapped around a portion of the user's anatomy. In some embodiments, the skin temperature monitor 112 measures body surface temperature of a user 101 in Celsius or Fahrenheit. In accordance with the principles of this disclosure, skin temperature data can be captured and processed in one or more file formats or file systems.

According to one embodiment, the biometric tracker 105 can further comprise a fMRI monitor 114 configured to measure, monitor, and map brain activity of the user 101. The fMRI monitor 114 can be provided in the form of an aspect of a fitness tracker, wearable device, a digital content playback device 115, or other portable device (e.g., movable scanning device) attached to a user's head, neck, arms, ro some other body part. In some embodiments, the fMRI monitor 114 can be provided in the form of a clinical device used to scan or otherwise monitor the user's 101 brain activity. In some embodiments, the fMRI monitor 114 detects variations in blood oxygenation and blood flow in portions of the user's brain using magnetic field(s), where the variations are detected in response to neural activity. In accordance with the principles of this disclosure, fMRI data 222 can be captured and processed in one or more file formats or file systems.

According to one embodiment, the biometric tracker 105 can further comprise a NIRS monitor 116 configured to monitor variations of oxygenated hemoglobin of the user 101. The NIRS monitor 116 can be provided in the form of an aspect of a fitness tracker, wearable device, a digital content playback device 115, or other portable device (e.g., movable scanning device) attached to a portion of the user's anatomy. In some embodiments, the NIRS monitor 116 can be provided in the form of a clinical device used to monitor a trait of the composition of the user's 101 anatomy (e.g., oxygenated hemoglobin concentration) using infrared light. In some embodiments, the NIRS monitor 116 detects variations in oxygenated hemoglobin concentrations and brain activity in portions of the user's 101 brain using near-infrared light, where the variations are detected in response to neural activity. In accordance with the principles of this disclosure, NIRS data 224 can be captured and processed in one or more file formats or file systems.

The digital content playback device 115 can be implemented as any type of digital content playback device, such as but not limited to a television, one or more monitors or other displays, speakers, VR headsets, headphones, adapted to playback immersive and/or flat digital content to a user. In some examples in which the digital content is 2D digital content such as video content, the digital content playback device 115 can be implemented as a television screen (e.g., a large flatscreen television) in front of which a user may be seated or stand to view the digital content.

In one particular example, the digital content playback device 115 can be implemented as any type of virtual reality device or other equipment for experiencing VR content, including but not limited to a headset that is worn over a user's eyes like a pair of goggles. The headset can block out external light and show a visual representation or image on one or more screens in front of the eyes. The view can be fully or partially immersive, providing a changing or static field of view in any direction the viewer chooses. The digital content playback device 115 can be coupled to third-party content 135, a digital content application 120, a smartphone application 130, or a computer interface 125. In some embodiments, the digital content playback device 115 can be provided in the form of a physical screen, such as one or more television screens. In such embodiments, the physical screen can include and/or be coupled to one or more speakers that playback sound or music related to the VR content generated in order augment the visual immersive experience of a user 101. The immersive media associated with the VR content can be monoscopic or stereoscopic 360-degree video or computer-generated environments assessed with a variety of psychophysiological monitoring methods, including but not limited to, the EEG monitor 102, the heart rate monitor 104, the respiratory 106, the blood pressure monitor 104, and the skin temperature monitors 112. The biometric data obtained from the monitors of the biometric tracker 105 while the user 101 is viewing digital content through the digital content playback device 115 can be used to intelligently train the iteratively trained training model 230 to generate new digital content and positively influence the user's experience with subsequent VR content that the user 101 becomes exposed to. In some examples, the iteratively trained training model 230 can also provide feedback to a user, such as feedback that indicates a user is getting stressed.

In some embodiments, the central controller 110 can be implemented as any computing device (e.g., a laptop, a desktop computer, a tablet, a server, a smartphone, a smart television, etc.) capable of being connected to the internet or other network (not shown). In some embodiments, the central controller 110 is implemented as a remote computing device (e.g., a server) and/or a cloud-based computing device. In some embodiments, the central controller 110 creates a monitoring and communication system between system components that is adaptable, scalable, reliable, and can accommodate different users, devices, manufacturers, and system configurations. In some embodiments, the central controller 110 is provided in the form of a data-processing device or system located proximate to one or more of the other components of the system 100 to collect, transmit, and receive data from the other system components. For example, in some embodiments, the central controller 110 can act as a network interface to create a communication link that operatively connects the biometric tracker 105 or the data therefrom, the digital content playback device 115, the digital content application 120, the computer interface 125, the smartphone application 130, and a user device (not shown). In another embodiment, the individual system components can also act as a network interface and create a communication module connection (not shown) directly to the other system components in a mesh-style network. In some embodiments, the central controller 110 is provided in the form of a data aggregator and operates in connection with the system architecture 200.

Furthermore, as one non-limiting alternative to the configuration seen in FIG. 2, the network can include a programmable processor 205 or a network interface and can be electronically coupled to a memory 210 or database device (e.g., a server). In some embodiments, the network can include program instructions that are stored on a cloud server non-transitory computer-readable medium and are executable by the programmable processor 205 to perform one or more of the processes described herein. In some embodiments, the processor 205, memory unit 210, power supply, gateway node, and similar processing components can be integrated into the central controller 110 to implement the processing and data aggregation tasks of the processes described herein.

FIG. 2 shows a schematic representation of a system architecture 200 for utilizing digital content as therapeutic treatment for psychological conditions, psychiatric conditions, and/or other general fitness and/or wellness purposes in accordance with one embodiment of the present invention. The system architecture 200, which may implemented in conjunction with the system 100 described herein, can be adapted to process biometric datasets and recommend and/or generate tailored VR content, AR content, MR content, audio content, spatial audio content, video content, audiovisual content, or any combination thereof, (referred collectively to hereinafter as “digital content”) to a user 101. Moreover, the system architecture can further be adapted to analyze a biometric response of a patient or user 101 to the generated digital content. The system architecture 200 can be used to execute one or more program instructions in connection with one or more methods for processing biometric datasets to generate digital content in the therapeutic treatment of specified psychological, psychiatric, or other medical conditions, as described in greater detail below.

In the illustrated example of FIG. 2, the system architecture 200 includes a backend processor 205, the central controller 110, and the memory 210. Although shown as separate components, in some embodiments, the processor 205 and/or the memory 210 are integrated and/or included within the central controller 110. In that regard, functionality described herein with respect to the processor 205 can also be attributed to and/or implemented directly by the central controller 110. The central controller 110 can be connected to one or more of the processor 205, the memory 210, a digital content experience module 225, an iteratively trained training module 230, a user biometric database module 235, and/or a biometric reference database module 240. Similarly, the processor 205 can be connected to one or more of the central controller 110, the memory 210, the digital content experience module 225, the iteratively trained training module 230, the user biometric database module 235, and/or the biometric reference database module 240

It will be recognized by one skilled in the art that the control system architecture 200 configuration could include computing devices in various configurations. The computing elements of the system architecture 200 can be provided via one or more computing devices that can be arranged, for example, in one or more server banks or computer banks, or other arrangements. Such computing devices can be located in a single installation or can be distributed among many different geographical locations. For example, the system architecture 200 can include computing devices that together can include a hosted computing resource, a grid computing resource, or any other distributed computing arrangement. The computing devices can further include one or more routers. In some cases, the system architecture 200 can correspond to a dynamic computing resource where the allotted capacity of processing, bandwidth, storage, or other computing-related resources can vary over time.

In a non-limiting example, the processor 205 can implement one or more lookup modules provided in the form of hardware, firmware, software, or a combination thereof. The lookup module(s) can be, for example, provided in the form of program instructions that can be executed by the processor 205. In some embodiments, the program instructions can be executed directly by the central controller 110. The lookup modules can include, in a non-limiting example, the datasets related to the biometric data stored in memory 210 or in one or more other databases or similar.

The memory unit 210 can be adapted to store biometric data, user data, data from the biometric tracker 105, or other data or information received from the system components, applications, third-party system(s), and sensing devices. The memory unit 210 can be implemented as a stand-alone memory unit or as part of the processor 205, part of the central controller 110, and/or part of some other aspect of the system 100.

In some embodiments, the system 100 receives biometric data from the biometric tracker 105 and stores the received biometric data in memory 210. This biometric data can include, but is not limited to, EEG data 212, heart rate data 214, respiratory data 216, blood pressure data 218, skin temperature data 220, fMRI data 222, and NIRS data 224. In some embodiments, the biometric data can be collected from one or more of the EEG monitor 102, heart rate monitor 104, respiratory monitor 106, blood pressure monitor 108, skin temperature monitor 112, fMRI monitor 114, or NIRS monitor 116, as described in connection with FIG. 1. In some embodiments, the biometric data stored in memory 210 or otherwise processed by the system 100 can be received as a data input, for example through data crawling, file uploads, manual entry, synchronization with a monitoring device, automatic updates, or other forms of data input into the system 100.

The system 100 can be used to process one or more aspects of the biometric data to tag, train, recommend, and/or generate digital content using one or more of the advanced analytics modules shown in FIG. 2, including but not limited to the digital content experience database module 225, the iteratively trained training module 230, the user biometric database module 235, and the biometric reference database module 240. In some embodiments, the system 100 generates digital content using generative artificial intelligence, or similar data processing techniques. In some embodiments, the system 100 and/or the system architecture 200 can include additional modules for processing and analyzing different data elements and data sets and are not limited to the data processing modules shown in FIG. 2. For example, the system architecture 200 can include any suitable number of processing modules (including a single processing module) for executing or carrying out any of the processes discussed herein.

The digital content experience database module 225 is designed to perform data processing techniques and generate digital content. The digital content experience database module 225 can also store digital content, including content created by the digital content experience database module 225, and/or third-party digital content, and/or other types of digital content or aspects thereof that can be played back for, or presented to, a user 101. In a non-limiting example, the system 100 can be used to determine physiological data and/or biometric data related to the user 101 while the user 101 views digital content. The system 100 can associate aspects, features, and/or other parameters of the digital content with readings from the biometric data. The biometric data can be evaluated with timestamps of segments of the digital content to determine patterns related to the user's 110 brain wave patterns and other physiological responses. Furthermore, the biometric data can be used to train the iteratively trained training module 230 to determine the user's 110 physiological response to new digital content and/or to generate new digital content to achieve a specific physiological response (e.g., relaxation state, focus state, etc.).

In at least this way, the present invention is an improvement over the prior art, as the system 100 leverages advanced data processing techniques to analyze biometric data indicative of the response of a user 101 to specific aspects of digital content, develops a predictive model (i.e., the iteratively trained training module 230) to score content watched by the user 101, wherein the score is developed based on the user's 101 physiological response as determined by comparing the user's biometric data to the user's historical biometric data and baseline biometric data insights from a population of users and digital content. Further, the system 100 can generate a score for new content based on the output of the iteratively trained training module 230 and generate new content predicted to impact one or more of the user's 101 biometrics (e.g., lower the user's heartrate) based on the data insights. The system 100 can also generate and present a predicted score to the user 101. For example, the system can present the predicted score to the user 101 via the digital content playback device 115, the digital content application 120, and/or one or more third-party applications. The predicted score, which is unique to the user 101, can indicate to the user 101 what type of physiological response is likely to be triggered in the user 101 by watching the new digital content before the user 101 selects the content. The system 100 can also recommend digital content to the user based on user input. For example, in response to the user 101 selecting a desire to focus and have a productivity session, the system 100 can recommend digital content from the digital content experience database module 225 that is likely to generate specific brainwaves associated with productivity and focus based on an output of the iteratively trained training module 230 (e.g., by decreasing the ratio of theta/low beta brain waves).

In some embodiments, the system 100 can upload digital content to a digital content categorization space (e.g., the digital content experience database module 225 in some examples) and the digital content can include a tag or other identifier associated with the physiological response(s) impacted by the digital content. In some embodiments, the system 100 comprises a tool and/or an application programming interface (“API”) that can be implemented in digital content playback platforms, such as the digital content application 120, the digital content experience database module 225, and/or any third-party video and/or VR platforms. This tool and/or API can provide feedback to a user regarding how a particular piece of digital content will make the user feel, how the digital content is predicted to impact the physiology of the user, and/or some other effect that the digital content may have on the user. In some embodiments, digital content that has been uploaded to a digital content categorization space (e.g., the digital content experience database module 225 in some examples) can be searched according to a predicted impact that the content will have on the user 101.

In some embodiments, the system 100 can further generate an impact score and transmit or otherwise display the impact score to a content creator. The system 100 can also be used to allow a content creator to upload content before it is displayed to the user. Then, the system 100 can process the uploaded content and provide a predicted impact score, insights to anticipated variations to physiology, and recommendations for how to improve the impact score (e.g., modify the color, texture, audio, shape, storyline, etc.). The system 100 can further generate recommendations tailored to specific physiological tags or identifiers in some embodiments (e.g., recommend changing the color to reduce heart-rate or to change the storyline to induce a calming effect, etc.). In some embodiments, the system 100 can include a search engine and/or a search feature included in a digital content categorization space (e.g., the digital content experience database module 225, the digital content application 120, third-party video and/or VR platforms, etc.) that allows users 101 and/or content creators to search for and/or select digital content according to the determined impact scores for the digital content.

In some embodiments, the impact score for a particular piece of digital content is calculated individually on the basis of an individual user's 101 bio data reaction to the particular piece of digital content. In that regard, there is not one impact score for a piece of digital content because the brains and hears of respective users do not always, if ever, have the same reaction to particular pieces of digital content. Much like prescription lenses, a piece of digital content has different refractories that can solve for unhappiness by boosting gamma asymmetry in the brain. Whether that “lens” works for an individual user depends on the particular biochemistry, or “mind's eye” of the individual user's brain.

The system and methods described herein are also an improvement over existing technologies in at least that the system provides greater access to treatment and therapeutic intervention since the system can be accessed by a user as much as they want without face-to-face treatment by a clinician, which requires transportation, financial resources, time, and an associated provider. The system and methods described herein can provide a user with an immediate and impactful reduction in acute anxiety and can be helpful for those with GAD as they experience acute bouts of anxiety, hypertension, or other conditions.

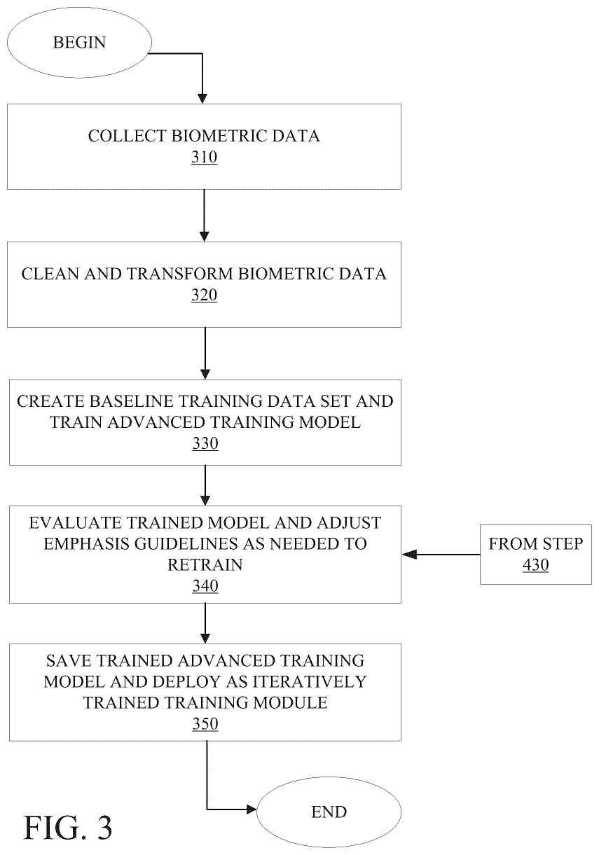

The iteratively trained training module 230 can be adapted to perform various data analysis and modeling processes. In one example, the iteratively trained training module 230 generates and iteratively trains training modules for providing dynamic outputs. For example, in some embodiments the training module 230 can be designed to perform one or more of the various data processing, comparing, determining, and configuring steps of the processes shown and described in connection with FIGS. 3-7. The training module 230 can be designed to generate, train, and execute nodes, neural networks, gradient boosting algorithms, mutual information classifiers, random forest classifications, and other machine learning and artificial intelligence-related algorithms.

The iteratively trained training module 230 is trained based on the biometric data inputs and parameters, features, or other aspects of the digital content being experienced by the user 101 at the time the biometric data is received. For example, the system 100 is configured to train an advanced artificial intelligence model to generate digital content to adjust a user's biometric data to replicate a state of relaxation, focus, positivity, or mindfulness. In some embodiments, the system 100 can be used to process third-party content 135, and determine timestamps associated with information from the content based on specific values in the user's 101 biometric data and further process that information to refine the iteratively trained training module 230. For example, if a user 101 is using the digital content device 115 to watch a third-party digital content or a VR experience, and the heart rate and/or respiratory rate of the user 101 spike, as detected by the heart rate monitor 104 and respiratory monitor 106 of the biometric tracker 105, the system 100 can capture the time of the sensed metrics. The system 100 can then compare that time to a timestamp in the third-party content and process that series of frames or segment of content for additional information to better train the model. The series of frames or segment of content can be identified, tagged, evaluated, scored, or rated based on its various parameters and the impact it had on the user's biometric data.

The system 100 can also be used to predict how a new piece of digital content will impact a user's 101 biometric data or emotional state based on the output of the trained model. In at least this way, with predictive analytics, the system 100 can objectively evaluate and determine a score for the digital based on a previous viewing history and/or associated biometric data of a user 101, to provide customized recommendations to the user 101 on the potential impact to their physiology or feeling state. In some examples, the system 100 can implement predictive analytics to determine a score for the digital content based solely current values of the biometrics of a user 101.

In some embodiments, the impact scores for respective pieces of digital content stored in a digital content library and/or a digital content database (e.g., the digital content experience database module 225) can be categorized according to how the digital content impacts the physiology of a particular user 101. For example, similar to how lens for prescription eyeglasses are categorized according to the impact of the lenses on a user's vision, digital content impact scores can be categorized according to how the digital content impacts one or more physiological parameters of a user. In some examples, a digital content library and/or digital content database can store particular subsets of digital content that are categorized according to respective impacts on particular user biometrics. For example, a first digital content library and/or database may store different pieces of digital content that can be played back to a user to correct for fast beta activity, a second digital content library and/or database that includes different pieces of digital content that can be played back to a user to boost positivity or left frontal gamma activity in the brain of a user, a third digital content library and/or database that includes different pieces of digital content that can be played back to a user to reduce high blood pressure, and so on. Within each of these respective digital content libraries, a “prescription value” (e.g., impact score) can be attributed to a respective piece of digital content based on the particular physiological and/or psychological impact that the digital content will have on a user.

The user biometric database module 235 can be adapted to receive data sets from the sensing and monitoring devices of the connected system 100, including the biometric tracker 105. In some embodiments, the user biometric database module 235 is further designed to compare the data sets to pre-defined threshold values, which are stored in the biometric reference database module 240. In some embodiments, the data sets compared by the user biometric database module 235 include the biometric data and the user data (e.g., age, health conditions, and other parameters) but can include any datasets, or other combination of information, from one or more databases and/or the memory 210.

In some aspects, the system 100 can use biometric data inputs from the biometric tracker 105 or from external data sources (e.g., a patient's electronic health records (EHR) or clinical test results) as input to the iteratively trained training module 230. In one non-limiting example, the system can receive fMRI data 222 and/or NIRS data 224, from a clinical setting or from the biometric tracker 105. The system 100 can be used to determine variations in the fMRI data 222 and/or the NIRS data 224 to provide recommendations to modify the digital content to impact the user's physiology in a specific way. In some embodiments, a user 101 can be viewing a piece of digital content that is being played back by the digital content application 120 (e.g., a video platform, a digital content streaming service, etc.) and the system 100 can alert the user 101 (for example, within the digital content application 120) that the user's 101 biometrics have strayed from a preset baseline.

In some embodiments, the system 100 can further include a deployment module and a notification module (both not shown). The deployment module can be designed for efficiently distributing requests to one or more system components or to third-party systems. In another embodiment, the deployment module can also be designed for pushing or installing updates to the system components from other system components or other aspects of the connected system 100. In some embodiments, pushing updates from one system component to other system components is completed automatically, although manual updates can also be configured using the system and processes described herein.

The notification module can be designed for generating, creating, modifying, and logging events, system updates, component updates, schedule updates, etc. for the system architecture 200 and the overall system 100. The notification module can also generate, update, and push notifications and customized alerts to one or more devices or applications for the user 101 or a third party. In one embodiment, the notification module is designed to be dynamic, scalable, and integrated with each system component, third-party systems, user device, control device, or a combination thereof. In some embodiments, the notification module is designed to receive data sets from the data sources or applications and handle the notification generation and handling for the data sources and applications.

In some embodiments, the system 100 can further include a diagnostic module (not shown). The diagnostic module can communicate with the sensing device(s) and the central controller 110 to determine that a system component is malfunctioning or has generated an error, warning, maintenance reminder, or similar. If an error code is detected, the relevant data sets can be transmitted to the system architecture for additional analysis and processing by an advanced analytics module, or similar.

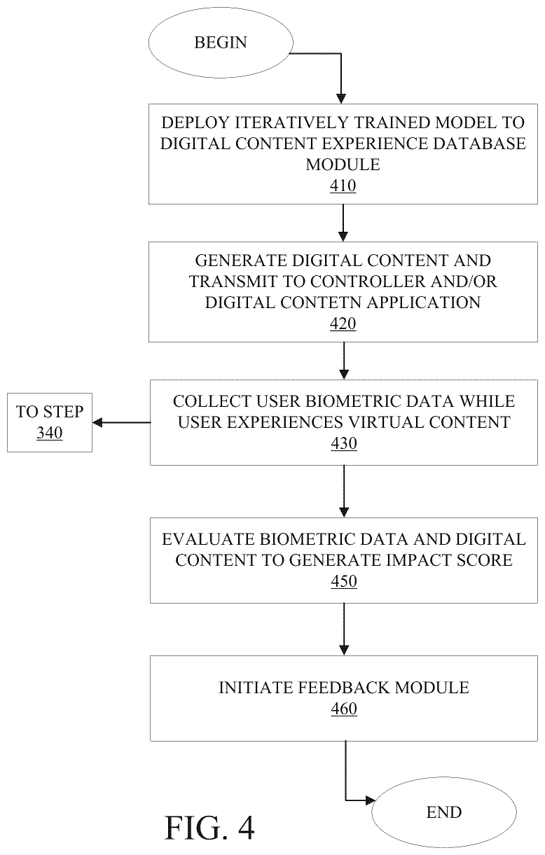

FIGS. 3 and 4 are flow diagrams illustrating the training and operation of one or more advanced training models, including those of the iteratively trained training module 230. Among other operations, the flow diagrams demonstrate a process for evaluating biometric data as an input for training an advanced training model. The process includes two main phases: processing datasets to train the advanced training module (FIG. 3) and deploying the iteratively trained training module 230 (FIG. 4).

A representative, non-limiting example of trained learning is shown in FIG. 3, in one embodiment, the advanced training model can be developed and iteratively trained to process biometric data or other datasets to determine a baseline dataset for training the advanced training model and detect patterns corresponding to a plurality of parameters related to digital content experienced by the user 101.

First, biometric data is received or otherwise collected in step 310. In some embodiments, the biometric data includes the data collected from the biometric tracker 105 as described in connection with FIG. 1. In some embodiments, the biometric data is collected while the user 101 is exposed to third party content. In some embodiments, the system 100 can be used to collect biometric data using a biometric tracker 105 of the system 100, or can process biometric data (or other information) received as an input to the system 100. In some embodiments, the biometric data can be pre-processed, filtered, or otherwise analyzed, edited, or processed at step 320 or throughout other steps of the processes described herein.

At step 330, the system 100 creates a baseline biometric dataset for training an advanced training model to detect patterns corresponding to a plurality of parameters associated with digital content. The advanced training model is trained to predict how the digital content affects the biometric data and feeling states of the user 101.

At step 340, the system 100 evaluates the accuracy of the advanced training model using a separate dataset from the training dataset. One or more emphasis values (e.g., weights, ranks, etc.) can be adjusted to modify the advanced training model and retrain.

At step 350, the trained advanced training model is saved and deployed in the system 100 as the iteratively trained training module and used to provide personalized recommendations or other insights based on the user's 101 biometric data, as described in more detail in connection with FIG. 4.

As shown in FIG. 4, the iteratively trained training module 230 can be used to generate digital content and/or recommend content that is predicted to induce certain feeling states or impact a user's 101 physiology in a particular way. At step 410, the iteratively trained training module 230 is deployed in connection with the digital content experience database module 225 to personalize digital content based on the user 101 biometric data. In some embodiments, the iteratively trained training module 230 can be used to predict how other content (e.g., third-party content) will affect a user's 101 biometric data based on the color, texture, shape, lighting, story narration, audio pitch, or sound of the digital content. In such embodiments, third-party content creators and/or providers can upload their content to a database, digital content platform, and/or some other tool that can be accessed by the iteratively trained training module 230. In that regard, the iteratively trained training module 230 assigns an impact score to the user uploaded digital content and/or the third-party digital content that indicates how the digital content will affect a user 101 (e.g., give the digital a score indicative of the amount of anxiety digital content will induce, the amount of happiness the digital content will induce, etc.).

At step 420, the system 100 generates digital content including, but not limited to an immersive VR experience as an output of the iteratively trained training module 230. Further, at step 420, the generated content can be transmitted to the central controller 110 to be displayed on one or more Digital content devices 115. In some embodiments, the central controller 110 generates the digital content using the Digital content application 120 before the content is displayed on the Digital content device 115. In some embodiments, the system 100 creates, generates, and deploys one or more surveys for the user 101 to complete before and after viewing the digital content. In some embodiments, the one or more survey can include a questionnaire and/or symptom checklist as described in U.S. Pat. Nos. 11,101,031 and 10,347,376, both incorporated by reference herein.

In some examples, at step 420, the system 100 can modify and/or generate new versions of previously uploaded third-party digital content. For example, the system 100 can modify a particular piece of third-party digital content by changing one or more of the color, texture, shape, volume, or some other feature of the third-party digital content to achieve a desired effect (e.g., reduce anxiety, reduce blood pressure, etc.) that playback of the third-party digital content may have on a user 101.

At step 430, the system 100 collects biometric data of the user 101 through the biometric tracker 105 or using a tracker external to the system 100 (e.g., user's personal fitness tracker, in a non-limiting example). In some embodiments, the system 100 can include a post-experience survey that is displayed after the user 101 finished viewing the digital content. The collected biometric data and the user's survey response is evaluated to iteratively train and improve the model at step 340 of FIG. 3.

The information and data collected for an individual digital content experience session can be used by the system 100 to generate an impact score at step 450. In some embodiments, the system 100 generates the impact score based on detected shifts (typically percentage shifts) in brainwaves (Hz), heart rate, skin conductance, blood pressure, or other biometric data, as evaluated by the iteratively trained training module 230. The iteratively trained training module 230 can further evaluated shifts in biometric data associated with specific aspects of the digital content by matching timestamps and similar segment identification elements.

At step 460, the user's 101 biometric data, survey inputs, and impacts score can be used as inputs to a feedback module (not shown) to continue to iteratively train the model and improve content generation and recommendations associated with third-party content.

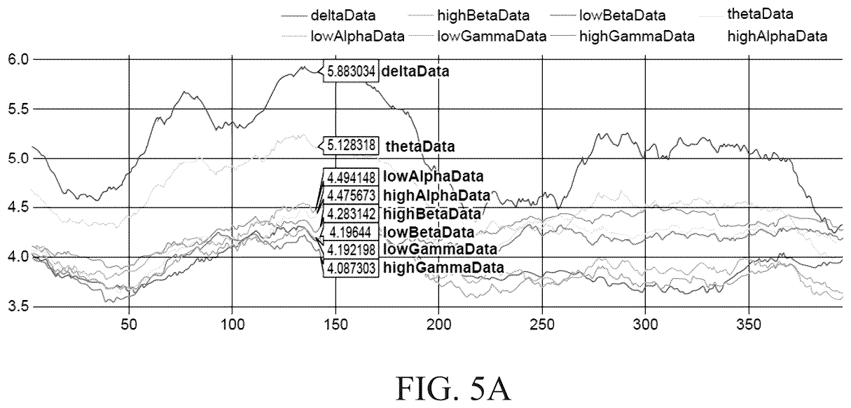

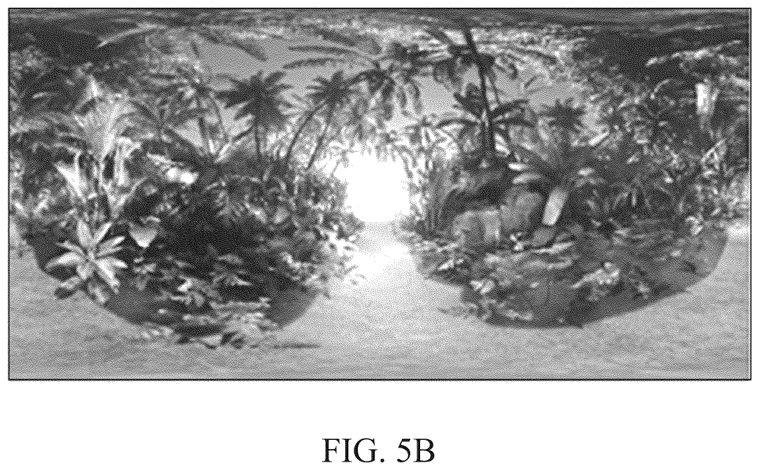

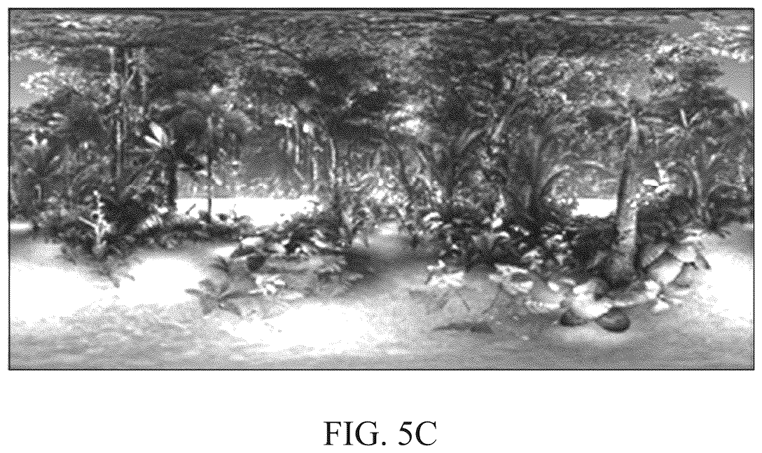

FIG. 5A is a non-limiting graphical illustrative example of EEG data 212 mapped to digital content, which can be used as an input to iteratively train the advanced training model and within the feedback loop used to iteratively train the iteratively trained training module 230. In the illustrative example shown in FIG. 5A, the voltages of recorded brain activity are graphically represented on the x-axis in Hz, and the time represented on the y-axis in seconds. In some embodiments, the time is associated with a timestamp of the digital content, such that one or more content parameters, (e.g., color, texture, shape, lighting, or sound of the digital content) can be extracted and associated with the biometric data as an input to the training model.

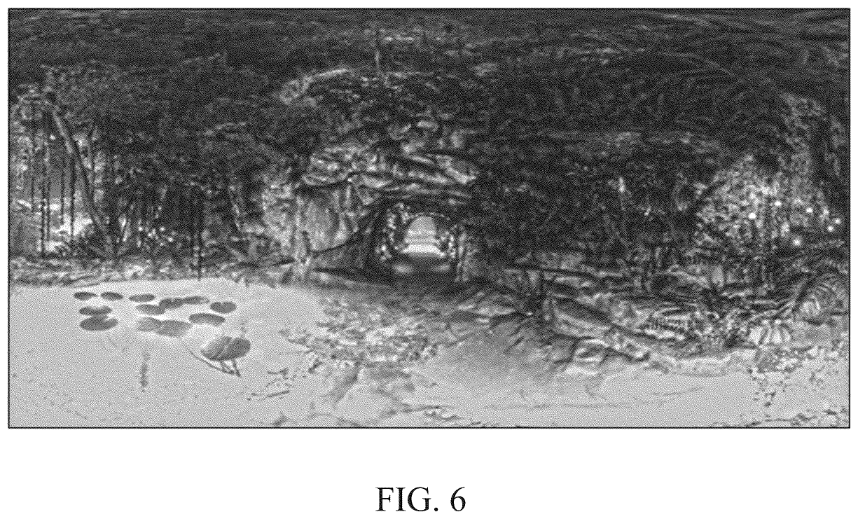

FIGS. 5B, 5C, and 6 represent screen captures of a digital content output, including a still image of a scene associated with an immersive VR experience generated by the system 100, by the processes described herein. In some embodiments, the digital content experience Database Module 225 can modify one or more parameters of the digital content to impact a user's 101 physiology. For example, the digital content experience database module 225 can modify color hues of a virtual scene, change an experience landscape, modify the experience weather, or other settings and features of the digital content in order to alter a user's 101 biometric data or feeling state. In some embodiments, the digital content can include on-screen instructions, such as “breathe in . . . breathe out” or a guided relaxation body scan to immerse the user 101 in a guided cognitive exercise to encourage the user 101 to notice how different parts of their body are feeling to them by “scanning” through aspects of their body one-by-one and shifting their focus to each body part. The biometric data collected in associated with the relaxation body scan, in this non-limiting illustrative example, can be used to provide data insights which can then be processed by the iteratively trained training module 230 to determine if there are specific aspects of the user's 101 body that are providing higher levels of acute anxiety and generate content based on the user's 101 response.

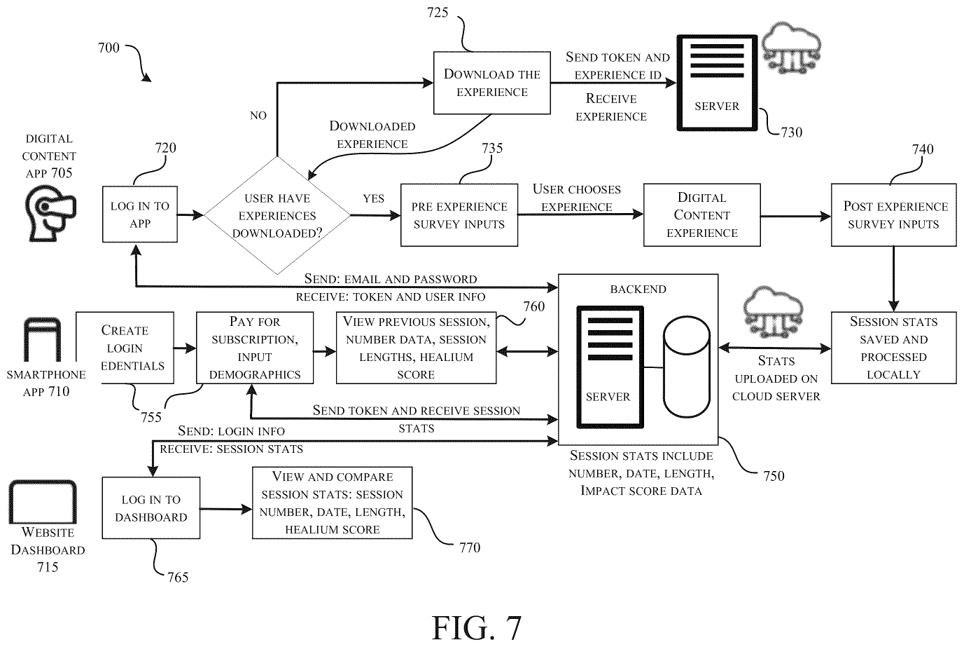

FIG. 7 is an alternative embodiment of the system architecture 700 that can be used to implement one or more of the methods described herein with respect to system 100. As shown in FIG. 7, the system 700 includes a digital content application 705, which is similar to the digital content application 120 described herein, a smartphone application 710, which is similar to the smartphone application 130 described herein, and a website dashboard 715. Alternative embodiments are also possible within the scope of the present disclosure. The digital content application 705, the smartphone application 710, and/or the website dashboard 715 can be implement on the same computing device (e.g., the central controller 110, the digital content playback device 115, a server, and/or some other computing) or one or more different types of computing devices.

In some embodiments, the digital content application 705 can include an authentication module 720 to verify a user's login credentials. After logging into the digital content application 705, a user can download digital content experiences 725 from a digital content server 730. For example, the digital content application 725 sends a token and/or a digital content experience ID to the content database server 730 to retrieve the digital content experience. The content database server 730 may be implemented as a cloud-based server. As further shown, the digital content application 705 can prompt a user 101 to input or otherwise receive feedback from a user 101 in the form of pre-digital content experience and post-digital content experience surveys 735, 740. The results of the surveys 735, 740 can be saved locally alongside digital content session stats 745 and/or saved remotely in a cloud-based backend server 750 with the digital content session stats (e.g., session date, number, length, impact score, biometric data, etc.).

In some embodiments, the smartphone application 710 can include an authentication module 755 for creating and verifying a user's 101 login credentials. Through this authentication module 755, a user 101 of the smartphone application 710 can also input a user's demographic information and/or other information about the user associated with a user profile, subscription requests, digital content experience requests, and/or other processes, features, and function. In some embodiments, the smartphone application 710 can include and/or use a session data module 760 for requesting and/or retrieving digital content session stats from the backend server 750.

In some embodiments, the website dashboard 715 can include one or more portals or windows, including but not limited to an authentication module 765 to verify a user's 101 login credentials, a user's demographic information and other information about the user associated with a user profile. In some embodiments, the website dashboard 715 can include and/or use a session data module 770 for requesting and/or retrieving digital content session stats from the backend server 750.

The system 100 can further comprise a statistics module for tracking and analyzing a user's sessions and biometric data associated with trends and historical information (e.g., specific VR experience session numbers, date, session length, content viewed, impact score generated for each VR session, etc.). In some embodiments, the biometric data collected and or the trends and evaluation data processed and analyzed by the system 100 can be used to generate tables, reports, or otherwise export the data and evaluation statistics for the user and/or a clinician.

In some embodiments, the iteratively trained training module 230 includes a neural network that has several layers. In one embodiment, the neural network is a multilayer neural network. In some embodiments, the input nodes represent inputs into the trained models (e.g., image data, metadata associated with the image data, etc.), one or more of the hidden nodes (e.g., one of the layers of hidden nodes) can represent one of the input vectors determined during the development of the detection model, and the output node represents the determined type of the content being analyzed.

In some embodiments, to iteratively train the one or more advanced training models, the system can compare a set of training outcomes from each of a plurality of training data sets and update one or more emphasis guidelines, classification values, characteristics, parameters, or similar. Additionally, in some embodiments, the comparison of the plurality of training data set outcomes can allow for the calculation of one or more error metrics between the input data and the output data. In at least one embodiment, the system can include a plurality of iteratively trained training modules 230, configured to generate outcomes, predictions, or classifications based on a particular data element characteristic. In some embodiments, the particular data element characteristic can include parameters related to the digital content, a specific feature, the type of content, or one or more user considerations (e.g., age, gender, medical conditions, etc.).

In some embodiments, the iteratively trained training module 230 model performs the iterative training of the one or more advanced training models. In some embodiments, the one or more iteratively trained training modules 230 can include an architecture with a certain number of layers and nodes, with biases and emphasis guidelines between the nodes. During training, the training module can determine the values of parameters weights and biases) of the machine learning model, based on a set of training samples. In one embodiment, the training module receives a training dataset for training. The training samples in the training set can include pre-existing digital content. For supervised learning, the training dataset can also include tags or labels for the aspects, features, or parameters of the digital content.

In an example of iterative training, a training sample is presented as an input to the iteratively trained training module 230, which then produces an output for a particular user based on the biometric data associated with the input training sample. The difference between the output of the iteratively trained training module 230 and a known output is used by the training module to evaluate and adjust the values of the parameters in the iteratively trained training module 230. This is iteratively repeated for a plurality of training samples to improve the performance of the iteratively trained training module 230.

The training module can also validate the trained the iteratively trained training module 230 based on additional validation samples. For example, the training module applies the iteratively trained training module 230 to a validation sample set to quantify the accuracy of the iteratively trained training module 230. The validation sample set can include digital content with associated known attributes. The output of the iteratively trained training module 230 can be compared to the known attributes of the validation sample set. In one embodiment, developing the iteratively trained training module 230 can include using validation data to compare to training data to determine if the iteratively trained training module 230 was being overfitted to the training data.
1. In some embodiments, a method for evaluating a biometric response of a user to digital content comprises the steps of collecting, via a biometric tracker, a first biometric data of the user, wherein the biometric tracker includes at least one sensing device; processing the first biometric data to establish a baseline dataset for the user; extracting at least one feature parameter from the digital content; collecting, via the biometric tracker, a second biometric data of the user while the user is exposed to the digital content; generating, via an iteratively trained training model, a model output based on the second biometric data and the at least one feature parameter; determining an impact score for the digital content based on the baseline dataset and the model output; and presenting, via a display device, the impact score to the user.
2. The method of clause 1, wherein the first biometric data and the second biometric data includes electroencephalogram (EEG) data.
3. The method of clauses 1 or 2, wherein the first biometric data and the second biometric data includes heart rate data.
4. The method of any of clauses 1-3, wherein the digital content comprises at least one selected from the group consisting of virtual reality content, augmented reality content, mixed reality content, audio content, spatial content, video content, and/or audiovisual content digital content.
5. The method of any of clauses 1-4, wherein the display device is a virtual reality device.
6. The method of any of clauses 1-5, wherein the at least one feature parameter can include one or more of a color, a texture, a shape, a lighting feature, a volume, a speed, a proximity, a location, or a sound of the digital content.
7. The method of any of clauses 1-6, wherein the impact score for the digital content is unique to the user.
8. The method of any of clauses 1-7, further comprising receiving third-party digital content from a content database; generating, via the iteratively trained training module, a predicted impact score for the third-party digital content; and recommending the third-party digital content to the user based on the predicted impact score and the second biometric data.
9. The method of any of clauses 1-8, further comprising generating a modified version of the third-party digital content based on the predicted impact score; and presenting, via the display device, the modified version of the third-party digital content based on the predicted impact score.
10. The method of any of clauses 1-9, wherein generating the modified version of the third-party digital content includes altering a story arc of the third-party digital content.
11. In some embodiments, a system for collecting evaluating biometric data from a user while the user is exposed to digital content comprises a biometric tracker including a sensor, wherein the biometric tracker is designed to collect biometric data of the user; a device adapted to present the digital content to the user; and a controller including a processor configured to process a first biometric data of the user collected by the biometric tracker to establish a baseline dataset for the user; extract at least one feature parameter from the digital content; collect a second biometric data of the user collected by the biometric tracker while the user is exposed to the digital content; process the second biometric data and the at least one feature parameter using an iteratively trained training module; determine an impact score for the digital content based on the baseline dataset and an output of the iteratively trained training module; and present, via the device, the impact score to the user.
12. The system of clause 11, wherein the biometric data includes one or more of EEG data, heart rate data, respiratory data, blood pressure data, functional magnetic resonance imaging data, near-infrared spectroscopy data, or skin temperature data.
13. The system of clauses 11 or 12, wherein the biometric tracker includes one or more of an EEG monitor, a heart rate monitor, a respiratory monitor, a blood pressure monitor, or a skin temperature monitor.
14. The system of any of clauses 11-13, wherein the processor is further configured to receive, from the user, a target physiological state; generate, based on the impact score and the target physiological state, a modified version of the digital content; and present, via the device, the modified version of the digital content to the user.
15. The system of any of clauses 11-14, wherein the at least one feature parameter can include one or more of a color, a texture, a shape, a lighting feature, a volume, a speed, a proximity, a location, or a sound of the content.
16. The system of any of clauses 11-15, wherein the digital content is two-dimensional video content; and wherein the device is a television.
17. The system of any of clauses 11-16, wherein the processor is further configured to receive, from the user, a target psychological state; determine a difference between a current psychological state of the user and the target psychological state based on the second biometric data; retrieve, from a digital content database, therapeutic digital content based on the difference between the current psychological state of the user and the target psychological state; and recommend the therapeutic digital content to the user.
18. The system of any of clauses 11-17, wherein the therapeutic digital content is stored in association with a predicted impact score.
19. The system of any of clauses 11-18, wherein the processor is further configured to determine the predicted impact score for the therapeutic digital content based on historical biometric data associated with the user.
20. The system of any of clauses 11-19, wherein the impact score for the digital content is unique to the user.

Various embodiments of systems, devices, and methods have been described herein. These embodiments are given only by way of example and are not intended to limit the scope of the invention. It should be appreciated, moreover, that the various features of the embodiments that have been described can be combined in various ways to produce numerous additional embodiments. Moreover, while various materials, dimensions, shapes, configurations, locations, etc. have been described for use with disclosed embodiments, others besides those disclosed can be utilized without exceeding the scope of the invention.

Persons of ordinary skill in the relevant arts will recognize that the subject matter hereof can comprise fewer features than illustrated in any of the individual embodiments described above. The embodiments described herein are not meant to be an exhaustive presentation of how the various features of the subject matter herein can be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The numerical ranges in this disclosure are approximate, and thus can include values outside of the range unless otherwise indicated. Numerical ranges include all values from and including the lower and the upper values, in increments of one unit, provided that there is a separation of at least two units between any lower value and any higher value. These are only examples of what is specifically intended, and all possible combinations of numerical values between the lowest value and the highest value enumerated, are to be considered to be expressly stated in this disclosure.

As used herein, “a,” “an,” or “the” can mean one or more than one. For example, “an” image can mean a single image or a plurality of images.

The term “and/or” as used in a phrase such as “A and/or B” herein can include both A and B; A or B; A (alone); and B (alone). Likewise, the term “and/or” as used in a phrase such as “A, B, and/or C” can include at least the following embodiments: A, B, and C; A, B, or C; A or C; A or B; B or C; A and C; A and B; B and C; A (alone); B (alone); and C (alone).

As used herein, the term “about” when referring to a measurable value such as an amount, a temporal duration, and the like, can include variations of +/−20%, more preferably +/−10%, even more preferably +/−5% from the specified value, as such variations are appropriate to reproduce the disclosed methods and systems.

As used herein, “statistical measures” can include the mean, the median, a percentile value, a variance, a standard deviation, or similar statistical measures, such as additional measures that can be derived from the above.

As used herein “color values” can include hue, saturation, lightness, red value, green value, blue value, magenta value, cyan value, yellow value, Lab space value, L*a*b* space value, or any other value derived from obtaining images from specific spectral regions, and which can involve the comparison of such values.

The constructions described in the accompanying materials and illustrated in the drawings are presented by way of example only and are not intended to limit the concepts and principles of the present invention. Thus, there has been shown, and described several embodiments of a novel invention. As is evident from the description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “can include” and not as “required.” Many changes, modifications, variations, and other uses and applications of the present construction will, however, become apparent to those skilled in the art after considering the specification and the accompanying drawings. All such changes, modifications, variations, and other uses and applications which do not depart from the spirit and scope of the invention are deemed to be covered by the invention which is limited only by the claims which follow.

What is claimed is:

1. A method for evaluating a biometric response of a user to digital content, the method comprising the steps of:
collecting, via a biometric tracker, a first biometric data of the user, wherein the biometric tracker includes at least one sensing device;
processing the first biometric data to establish a baseline dataset for the user;
extracting at least one feature parameter from the digital content;
collecting, via the biometric tracker, a second biometric data of the user while the user is exposed to the digital content;
generating, via an iteratively trained training model, a model output based on the second biometric data and the at least one feature parameter;
determining an impact score for the digital content based on the baseline dataset and the model output; and
presenting, via a display device, the impact score to the user.

2. The method of claim 1, wherein the first biometric data and the second biometric data includes electroencephalogram (EEG) data.

3. The method of claim 1, wherein the first biometric data and the second biometric data includes heart rate data.

4. The method of claim 1, wherein the digital content comprises at least one selected from the group consisting of virtual reality content, augmented reality content, mixed reality content, audio content, spatial content, video content, and/or audiovisual content digital content.

5. The method of claim 1, wherein the display device is a virtual reality device.

6. The method of claim 1, wherein the at least one feature parameter can include one or more of a color, a texture, a shape, a lighting feature, a volume, a speed, a proximity, a location, or a sound of the digital content.

7. The method of claim 1, wherein the impact score for the digital content is unique to the user.

8. The method of claim 1, further comprising:
receiving third-party digital content from a content database;
generating, via the iteratively trained training module, a predicted impact score for the third-party digital content; and
recommending the third-party digital content to the user based on the predicted impact score and the second biometric data.

9. The method of claim 8, further comprising:
generating a modified version of the third-party digital content based on the predicted impact score; and
presenting, via the display device, the modified version of the third-party digital content based on the predicted impact score.

10. The method of claim 9, wherein generating the modified version of the third-party digital content includes altering a story arc of the third-party digital content.

11. A system for collecting evaluating biometric data from a user while the user is exposed to digital content, the system comprising:
a biometric tracker including a sensor, wherein the biometric tracker is designed to collect biometric data of the user;
a device adapted to present the digital content to the user; and
a controller including a processor configured to:
process a first biometric data of the user collected by the biometric tracker to establish a baseline dataset for the user;
extract at least one feature parameter from the digital content;
collect a second biometric data of the user collected by the biometric tracker while the user is exposed to the digital content;
process the second biometric data and the at least one feature parameter using an iteratively trained training module;
determine an impact score for the digital content based on the baseline dataset and an output of the iteratively trained training module; and
present, via the device, the impact score to the user.

12. The system of claim 11, wherein the biometric data includes one or more of EEG data, heart rate data, respiratory data, blood pressure data, functional magnetic resonance imaging data, near-infrared spectroscopy data, or skin temperature data.

13. The system of claim 11, wherein the biometric tracker includes one or more of an EEG monitor, a heart rate monitor, a respiratory monitor, a blood pressure monitor, or a skin temperature monitor.

14. The system of claim 11, wherein the processor is further configured to:
receive, from the user, a target physiological state;
generate, based on the impact score and the target physiological state, a modified version of the digital content; and
present, via the device, the modified version of the digital content to the user.

15. The system of claim 11, wherein the at least one feature parameter can include one or more of a color, a texture, a shape, a lighting feature, a volume, a speed, a proximity, a location, or a sound of the content.

16. The system of claim 11, wherein the digital content is two-dimensional video content; and
wherein the device is a television.

17. The system of claim 11, wherein the processor is further configured to:
receive, from the user, a target psychological state;
determine a difference between a current psychological state of the user and the target psychological state based on the second biometric data;
retrieve, from a digital content database, therapeutic digital content based on the difference between the current psychological state of the user and the target psychological state; and
recommend the therapeutic digital content to the user.

18. The system of claim 17, wherein the therapeutic digital content is stored in association with a predicted impact score.

19. The system of claim 18, wherein the processor is further configured to determine the predicted impact score for the therapeutic digital content based on historical biometric data associated with the user.

20. The system of claim 11, wherein the impact score for the digital content is unique to the user.
